(19) United States
(12) Patent Application Publication
(10) Pub. No.: US 2003/0149344 A1
(43) Pub. Date: Aug. 7, 2003

(54) Applications of the biofeedback technique and cardio vascular monitoring

(76) Inventor: Yaniv Nizan, Modiyn (IL)

Correspondence Address: KATTEN MUCHIN ZAVIS ROSENMAN, 575 MADISON AVENUE, NEW YORK, NY, 10022-2585, US

(21) Appl. No.: 10/367,564

(22) Filed: Feb. 14, 2003

Related U.S. Application Data
(63) Continuation of application No. PCT/IL01/00749, filed on Aug. 13, 2001.
(60) Provisional application No. 60/300,810, filed on Jun. 27, 2001.
(60) Provisional application No. 60/231,849, filed on Sep. 12, 2000.
(60) Provisional application No. 60/225,652, filed on Aug. 16, 2000.

(51) Int. Cl. A61B 5/00

(52) U.S. Cl. 600/300

(57) ABSTRACT

Psycho-physiological sensors are used and the hand-held device it connects to pre-processes their measurements. The hand-held device might be a wire telephone and/or cellular telephone and/or PDA and/or wear-on computer. The measurements data can be pre-processed at the device before sent via the network to the server computer. The data intended to be used for control of games at the device and/or any other biofeedback display (i.e. visual display for the purpose of stress relief or blood-pressure control).

The use of the measurements in a networked environment where the data is sent to a central server, processing data of many users.

The use of the sensors' measurement of psycho-physiological parameters in order to control and operate devices usually controlled by pressing buttons and (i.e. Air-condition system, Refrigerator etc.).

### 1. TECHNICAL FIELD, BACKGROUND ART

In this document the term BioHHD will be used when refering to the hand-held device used by the used that might be a wire telephone and/or cellular telephone and/or PDA and/or wear-on computer

### BRIEF DESCRIPTION OF THE DRAWINGS

These and further features and advantages of the invention will become more clearly understood in the light of the ensuing description of a preferred embodiment thereof, given by way of example only, with reference to the accompanying drawings, wherein

FIG. 1 is a block diagram of a Biofeedback Application at Cellular Network according to the present invention;

FIG. 2 is a block diagram of Biofeedback for WLL (Wireless Local Loop) Network according to the present invention;

FIG. 3 is a diagram illustrating the Biofeedback IC principal according to the present invention;

FIG. 4 is a diagram illustrating the Biofeedback Application data-grams according to the present invention;

FIG. 5 is a diagram illustrating the Biofeedback at a BioHHD according to the present invention;

FIG. 6 is a diagram illustrating Implementation Example—BioHHD Cellular application according to the present invention; Implementation Example—BioHHD Cellular application

FIG. 7 is a flow illustrating the heart checkup analusis according to the present invention;

FIG. 8 is a diagarm of Theoretical basis of the proposed card logical testaccording to the present invention;

FIG. 9 is a diagram illustrating the heart checkup systsm and information flow according to the present invention;

FIGS. 10, 11, and 12 are flow chart illustrating the crdio analysis algotithm according to the present invention;

FIG. 13 is a diagram illustrating information analysis flow according to the present invention;

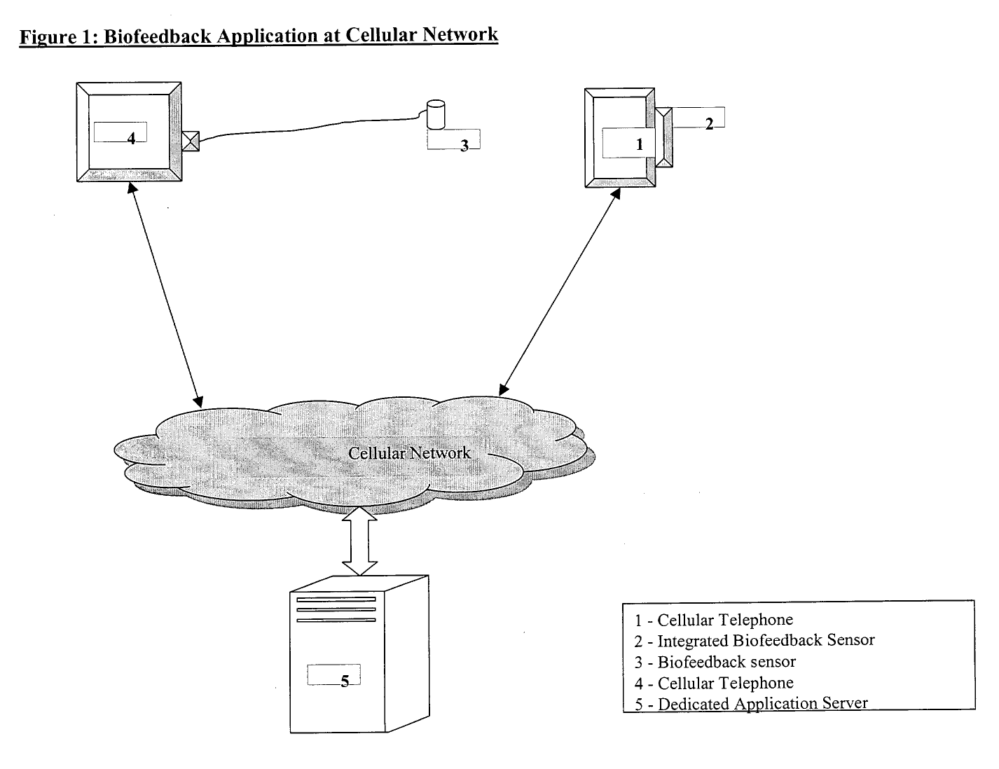

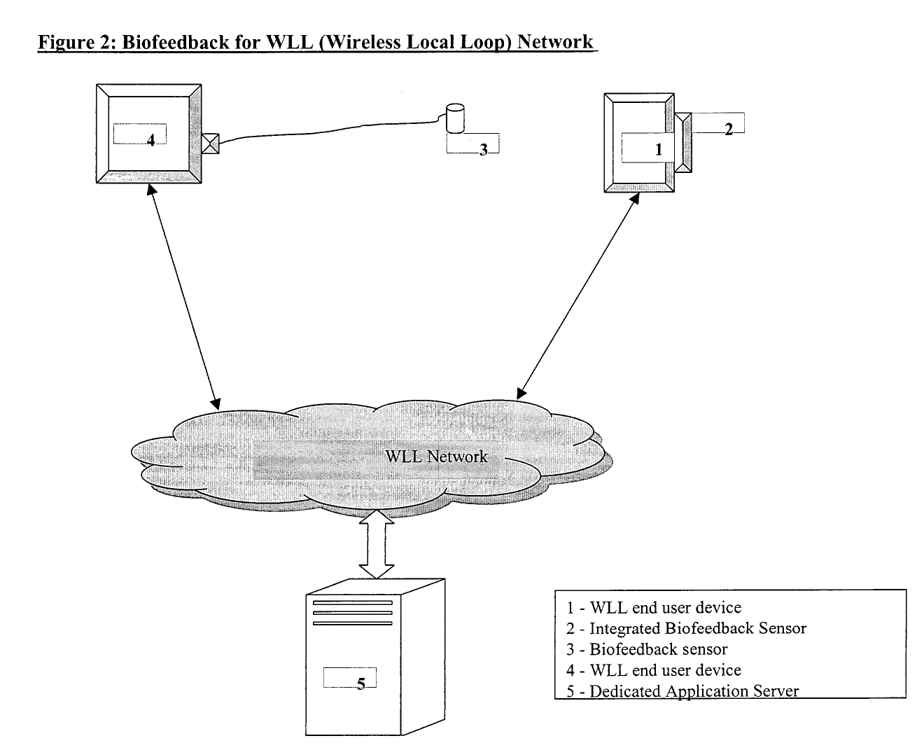

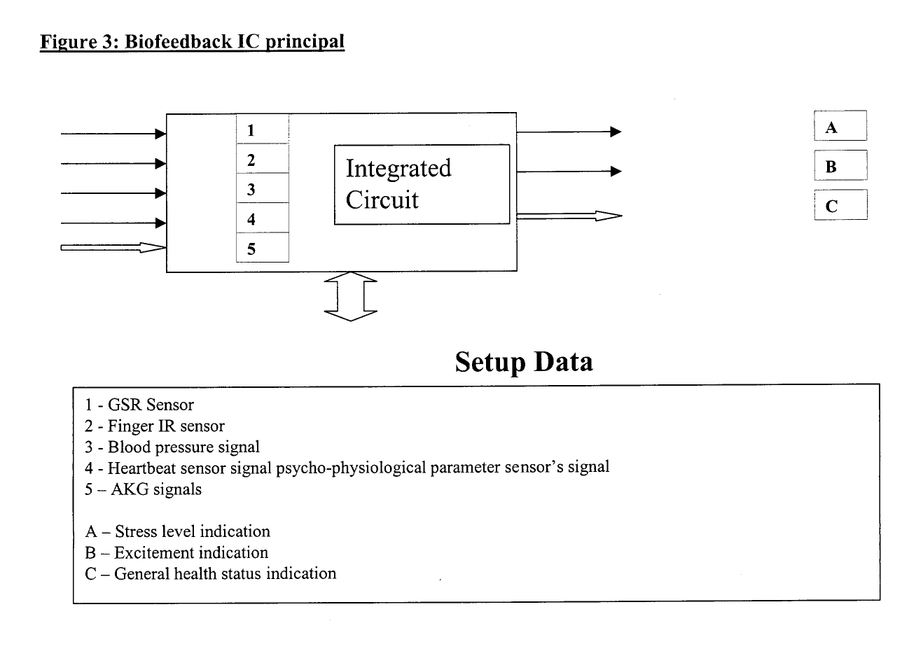

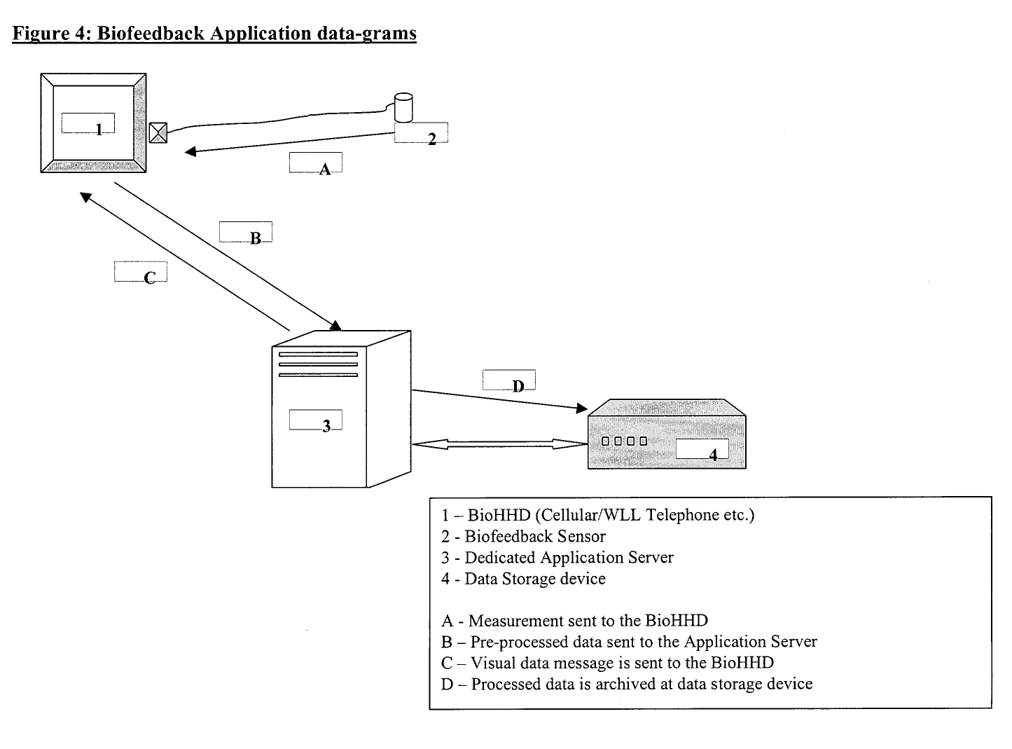

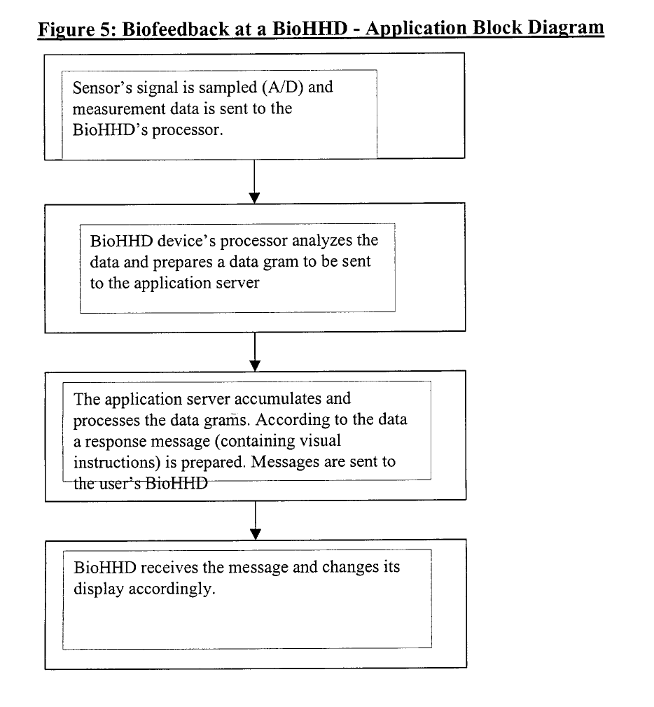

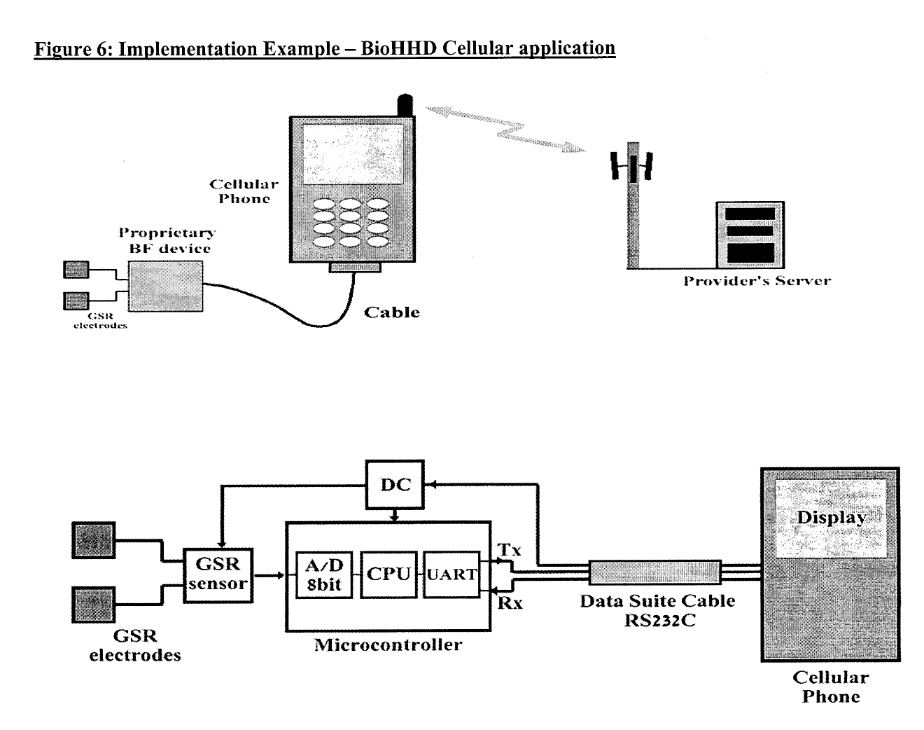

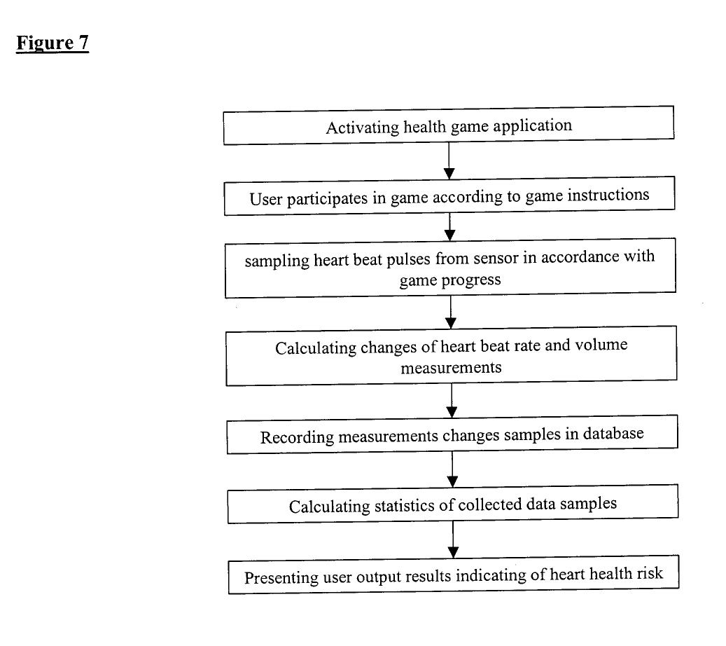

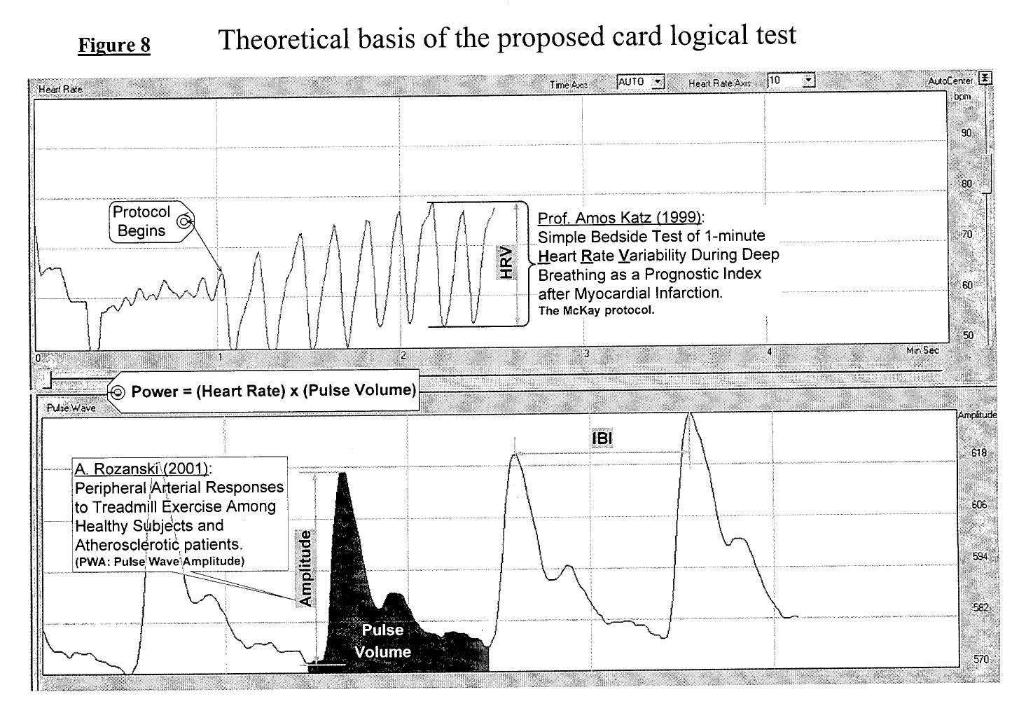

### DISCLOSURE OF INVENTION

A. This Proposal Focuses on Implementing the Basic Principles and Technologies of the Biofeedback Technique, Developed in a Clinical Context, to Improve Interaction with the BioHHD.

The basic principle of the biofeedback technique is that providing a person with feedback obtained by measuring mainly physiological signals from the skin of the fingers, and displaying them externally by simple visual means—usually light and sound, e.g. rising and falling tones—creates an open monitoring loop. This enables the study and modification of complex systems in the body, some of which are involuntary and non-autonomous.

This technique is currently being used in a clinical context by means of specially designed tools. The technique has existed in the clinical field—in medicine, psychology and rehabilitation—for decades. Scientific research recognizes the technique's efficiency. The necessary equipment and feedback methods are continually updated in accordance with innovations in entertainment electronics, and have been reduced to dimensions, which allow the creation of personal devices the size of a cellular telephone. Despite this, the technique is mainly used in large devices such as personal computers, to enable clear interaction between patients and therapists. As patients are not normally familiar with the behavior changes needed to bring about desired physiological changes, they require the aid of an experienced therapist, and a large monitor for joint study.

An exhaustive search of scientific, technical and patent databases has revealed that the biofeedback principle has not yet been implemented for daily interaction with applied electronics devices.

For further details on the biofeedback technique, visit the official site at aapb.org.

For an illustration of how interacting with the medium affects mental change, see the web site do-not-zzz.com.

B. To Render Biofeedback Principles Suitable for Developing Methods of Interaction with a BioHHD, we will Assume that:

1. People who do not suffer from special clinical problems do not require the physical presence of a therapist or anyone else in order to create a learning cycle using biofeedback tools.

2. Biofeedback tools can be implemented in a non-clinical context.

3. The various stages of the biofeedback-learning loop can incorporate feedback obtained from devices or people who are not physically present.

4. It is possible to implement only a part of the complete biofeedback loop, e.g. information only, information involving another device, information and device activation, or the complete biofeedback loop, incorporating behavior changes.

5. To render the biofeedback loop suitable for a BioHHD, standard human engineering principles should be followed, e.g. incorporating colors and sounds, which are changed from one desired state to another according to human feedback when the phone is being used.

C. To Determine the Possibilities of Rendering Biofeedback Principles Suitable for BioHHD Applications, we will Distinguish the Following:

1. The number of persons involved: the user only, the user and one other person, the user and numerous other users.

2. The number (small or large) of stages which can be implemented for such interaction on biofeedback principles: information only, information involving another device, information and device activation, or the complete biofeedback loop, incorporating behavior changes.

The Combinations of these Possibilities are Set Out in the Following Table:

| Stage of learning loop | Number of persons involved | | |
|---|---|---|---|
| | User | User and important other | User and group |

Feedback only
Feedback and learning

Such a table can determine the development and marketing process of the product from a “simple” to a “complex” cell, e.g. progressing from a product providing a simple function such as personal feedback to a complex one such as group learning.

D. Additional Medical and Psychological Principles Involved in Applying the Model to Home Consumer Products, such as a Refrigerator or Air-Conditioner

1) Principles from the Field of Psychology

The cost of the development, service and marketing of technology-rich products in the era of global economy, together with the ever-changing nature of the technology, create a high entrance-barrier for manufacturers. It obliges them to organize themselves in global groups. In order to reach the maximum number of clients, man/machine interfaces of the apparatuses get more and more limited. As a result of this, existing interfaces do not allow adjustment to be made for the sake of compatibility to different populations, and inter-activity. This situation is incompatible with the projection of good life in this era, a projection that is consistent with the intensifying of clients' perceptions along with a wider, more diversified range of demands. This situation brings about a sense of need for a more variegated, less uniform product.

Therefore, because of the limitations of the interface, exactly these apparatuses associated with welfare are causing a negative exchange between the values of stress and the ones of a good feeling and high quality of life. In order to achieve a marketing advantage, every manufacturer tries his best to achieve this advantage or to expand the market, by carrying out an additional, secondary exchange of values—in between other quality values such as: level of technology, dependability, styling, price—in coordination with the regular change-over being performed while establishing their marketing strategy, on the quality curve between technology to price. This balance carries with it a high marketing cost, and is done separately for every product. Along with the frequent changes in the fields of technology and fashion, resulting from the products' high marketing cost, these do not allow manufacturers to benefit from their global advantage in the market, and from their types of products. This means that actually, there exists a situation of balance between marketing profits and technological advantage. Since this exchange is inefficient, the need arises for an alternative to it.

Biofeedback is the hub of an inter-active model to be used in consumer apparatuses, which will bring about a more efficient exchange between technology and price on the quality curve. However, careful design and application of it is imperative, as detailed in the following paragraphs.

The general principle of such a design is, that as the consumer doesn't like to be consciously active, it is desirable to make use of every apparatus in simple combinations of light and sound to which the user is accustomed and culturally conditioned—for instance: control-displays on the refrigerator's door and the sound of opening that door. At a later stage it will be possible to give an output consisting of more complex, but still primary and natural, combinations of light/sound—for instance: fluorescent colors on a transparent refrigerator door. Only at the last stage there is to be made use of multimedia, which is not cybernetic.

2) Medical Principles:

The body's language is measured by such measures as: plentiful sweating, changes in the skin's degree of (electrical) conductivity, the intensity of the pulse, its compounding and quickness, body temperature, the blood pressure, the respiration: its rate, depth, level of blood oxidization, as well as reaction of the different skeleton muscles, such as contraction and relaxing.

These measures form together an expression of the activity of the autonomic systems of the body: the ortho- and para-sympathetic systems. These systems are required in order to react to the varying surroundings where the body may be at the moment, and adjust its responses at the level of the various systems, including the mental ones, so as to enable optimal continued functioning.

The degree a human being's ability to adapt to existing environments is dependent upon the structure of his individual personality, the sources of strength he had acquired, as well as on the intensity of the stimuli by which the surroundings are threatening to the individual. Thus, the adaptation ability is a personal factor, and is characterized by the quickness of response and the intensity of that response, which differ from one individual to another.

This is why it is so important to identify and devote the necessary consideration to the needs of the individual—whether by relating to the objective surrounding conditions around him, and adjusting them as far as possible to his ability, (including the referring to signs of mental stress), which are sometimes left unnoticed at the conscious level or denied.

From this stems the importance of recognizing any expressions of the autonomic systems and their meaning as a sensitive source of information about the perceptions and feeling of the person, at both the physical and mental levels. Through repeated measurements, taken under optimal conditions, it is possible to arrive at a statistical “average”, to which the response of the autonomic system—under differing conditions—is comparable, or even to make the comparison with reference to a general statistical “average”, which had been checked in many individuals.

While checking this “average” it is important to devote the necessary consideration to the existence, generally, of two different conditions of stress:

EUSTRESS: This state of excitement and of normal tension is under control. Example: when watching sports games, in an amusement park, etc.

DISTRESS: This is a condition of excitement, tension and a sense of lost control, a state of stress which is more difficult to neutralize. This is a very frequent state; characteristic of the complexity of modern lifestyle, and it expresses itself in the morbidity that comes with the modern style and way of life.

Changing the conditions in an objective environment according the reactions of the autonomic system of the individual, can be done in any site where the good feeling of the person is of utmost importance, which is every air-conditioned closed space. An extreme example of a site where the good feeling of the person is of utmost importance, is also paradoxically one of the places where a human being finds himself under many difficult pressures, both the ones that come from the surroundings as well as those stemming from psychological pressures: this is when flying a civilian, passenger-airplane; therefore, application of the product to the airplane is of high importance. A rather more ordinary, daily site is the office, where the individual spends most of the hours of his day, and where the highest degree of tension is formed, a tension that has a negative influence on the work output and on morbidity.

There are several indices which may be used, and these can be applied when determining the degree of suffering while designing products that utilize physiological signals for the purpose of adjusting an environmental apparatus, such as an air-conditioner, for the benefit of the user:

Number of breaths per minute: this expresses a good or bad psychical feeling, and is an expression of physical stress. This measure can be determined by the change which takes place in the pulse-wave (that would be measured by a pulse-microphone or blood-pressure), or by using a seat belt in which a tachometer is installed, and will determine rapid breathing and/or deep respiration;

Pulse;

Sweating at the finger-ends: sweat brings about a local change in skin conductivity, which indicates sympathetic activity linked to feelings;

Involuntary contraction of muscles as an expression of stress;

Extremely high or low blood-pressure;

Level of oxygen in the auricle of the ear or on the finger;

Temperature

When designing the sensors and forming their shape, it is desirable to integrate as many of the sensors as possible into ordinary habits: thus, in an example of the airplane, sensors should be used by integration into common accessories like headrest-earphones, seat handle, seat-belt, taking the shoes off and touching a pleasant floor-surface etc.

### 2. INDUSTRIAL APPLICABILITY

A. The Personal BioHHD will Act as a Terminal for Receiving Physiological Signals from the User's Body.

The BioHHD will be defined as a terminal for receiving physiological signals from the user's body by the technological channels currently in standard use in biofeedback involving personal computers. Such a terminal can be one of several types of BioHHD:

1) Line Telephone

A normal line telephone, connected to a communications network by a physical line. This device can be a stand-alone phone, or one integrated with another device, such as a fax or cordless phone.

2) Cellular Phone

A cellular or mobile phone functioning under all communications protocols: GSM, TDMA, etc. This device too can be stand-alone or integrated into another device, e.g. a desktop computer or a Palm Pilot.

3) Types of Input Methods

The telephone will be capable of receiving, separately or in parallel, the standard types of physiological feedback currently used for biofeedback in a personal computer. These are: GSR, EMG, skin surface temperature, heart rate, breath volume. (For further details on biofeedback channels and types of sensors for each channel, see the site at aapb.com.)

4) Location of Sensors

The physiological sensors can be located in various parts of the BioHHD or the connected device, such as:

A) On the handset: The sensors are integrated into the BioHHD handset. Since each biofeedback channel has its own electrode, the BioHHD can be provided with one electrode per type of channel.

The handset can be ergonomically designed: either to suit a human hand allowing better contact with the surface of the skin, or with holes or depressions in the handset for the user's fingers, so that sensors placed inside the depressions come into contact with the skin. Another possibility for ergonomic design of the handset is a concave shape, in which the user's chin comes into contact with the sensors.

B) At any other point on the exterior of the BioHHD:

For example, in electrode panels on the sides of the instrument, e.g. strips of sensors on the sides of the BioHHD, which the user's free hand can grip or rest on while the other hand holds the BioHHD.

As in the previous item, this system can be designed ergonomically to improve the quality of body signals: e.g. a raised or depressed panel for convenient holding while the phone is in use.

C) In another device:

The sensors can also be located in another device, directly connected to or integrated with the BioHHD, or one which transmits signals from the user to the BioHHD using a radio, telephone, or cellular communications protocol. For example:

A wristwatch transmitting pulse or perspiration data from the skin surface

An electrical household appliance which is installed with physiological finger sensors, such as a refrigerator handle fitted with sensors, and which transmits signals to the telephone locally using a radio, telephone, or cellular communications protocol.

An electrode bearing device carried on the body, e.g. an article of clothing fitted with such sensors, or a special purpose electrode strip similar to those used in medical examinations or in sports. Example: the inner portion of a belt buckle can carry a breath volume sensor, transmitting continuous data to the BioHHD by radio or infra. The belt, like the telephone, will have a “green” position, since breath is the function most impaired by stress generally and its indications such as cellular phones specifically.

D) Installation inside the body.

B. Development of Existing Software and Hardware for BioHHDs will Transform the BioHHD into a Local Real Time Processing Station for Physiological Signals Received from the Body.

The BioHHD hardware will incorporate a system allowing real time processing of the user's physiological input, in several ways:

1. Without Connecting to the BioHHD Network:

Special electronic components or chips, integrated into the BioHHD's electronic circuit, will independently process the physiological input in real time. Such process may or may not require activating the BioHHD. Data will be stored in the device as computer memory.

Data can also be stored as electronic memory on a smart card, which can be passed between BioHHDs.

2. By Connecting to the BioHHD Network:

A) The network will aid input processing automatically, without the user's having to perform any action.

B) The network will aid input processing with the participation of a human expert. E.g. a doctor or psychologist can observe the transcribed signals and help decipher them using general experience, or by comparison with the user's signal database, which is stored in the network.

C. OutPut

Once physiological signals have been received from the user and processed in real time, whether locally by the BioHHD or using a network if necessary, they will provide feedback in the following ways. The list below is a kind of ascending scale, from the aspects of: The feedback loop, Involvement of others in the feedback

1. Providing Feedback by Means of the BioHHD

When the user holds the cellular phone so that his or her fingers come into contact with sensors located on the phone, the device will analyze the user's mood and report it based on biofeedback principles. The user will receive this information by the usual biofeedback means, i.e. simple auditory or optic output. These principles will be adapted to the characteristics of the BioHHD. The visual display will change according to at least two principles: the input's motion or lack of motion in a desired direction, and passage from desired to undesired color combinations, according to standard principles for such combinations, e.g. Munsell's color solid. For example, a red and yellow color combination will constitute a “noisy” extreme of desired combination, while a blue and green combination will be a “calm” extreme, desirable for other users.

The auditory output produced by the device can constitute, for example, slow baroque style music at the “calm” extreme, and fast rhythms like trance music at the other extreme.

In this way, the user holding the BioHHD can be provided with different musical rhythms, screen colors, pictures or video clips, expressing his or her mood at that time, and can change this state using the BioHHD as a monitoring device.

Example: changing the color of a light bulb inside a transparent antenna to a calm color such as blue in peaceful states or to a less calm color such as yellow in stressful states.

Another example: A heart or smiley face on the device display could change its color or state according to the user's state of stress or calmness. This symbol could be programmed to show humorous messages or advice in case of an extreme mood shift.

2. Providing Feedback by Means of Another Device

By the same biofeedback principles, the BioHHD as a reception terminal can function separately from another device, which will be the feedback instrument. This device will be connected to the BioHHD using a communications protocol, such as radio, BioHHD, cellular, or internet.

Example: Feedback is provided by selecting a calm or stressful television channel, the type of music played by a home or computer audio system, the color or intensity of room lighting, air conditioning temperature, etc.

3. Feedback for Halting Behavior or Activity

When the BioHHD identifies stress or fatigue, and the user is not interested in an interactive feedback process but wishes to rest and recuperate, feedback will be used only to halt the activity the user is engaged in.

For example, if the same data pattern is received from two sensors in the BioHHD, showing a gradual decrease in skin temperature along with a rise in EMG, this may indicate that the user is nervous due to an event related to use of the BioHHD, such as information received. In such a case, the device will suggest in various ways that the user should limit the use of the BioHHD, by the standard biofeedback methods, i.e. auditory or visual output programmed into the device. For instance, a sad face might appear in the corner of the display to hint at the user's state of mind.

4. Feedback by Activating Appliances or Programs

In such cases, the feedback loop will utilize appliances or programs located inside the BioHHD, in the device with which it is integrated, or in a device connected to it by protocols as detailed.

For example, average upper and lower GSR thresholds for the entire population may be determined. These levels will be programmed into the device as a default. Each user will be able to adjust these levels according to his or her own data. If signals rise above a defined threshold in some physiological channel, for example as a result of accumulated stress, the BioHHD will automatically check the user's internet account for email or voice mail. This activity will serve to release stress and help calm the user. If calmness continues unconsciously to a point significantly lower than the upper threshold, the receipt of email will be approved.

Another example: The possibility of phoning a loved one when a physiological index considered emotional and internal, e.g. skin surface temperature, passes a threshold indicating a state of significant calm and intimacy, for instance a temperature of 34.5° C. measured on the palm during the working day. In this case, the device will suggest making a phone call to a loved one or sending a message, such as a preprogrammed heart symbol, when the index passes the threshold.

On the other hand, when the temperature index passes a lower limit indicating “coldness and emotional stress”, e.g. palm temperature drops below 23° C., the device will be programmed to activate an encouraging, cheering program, such as music or a game.

Another possibility is performing a predicted action in advance:

For example, if a user performs the same action, such as checking email or BioHHD messages, whenever feeling stressed as measured by GSR and ASR indices, and phones his or her spouse whenever feeling especially calm by the same indices, then the BioHHD's computer memory will learn this behavior pattern and predict it in future. When the relevant indices are measured, for example when the device is activated, it will suggest performing the action by means of a visual cue.

Biofeedback can also be used for correcting actions:

Correcting an action performed by the BioHHD by using biofeedback received from the user concerning this action through the sensors installed on the device. The BioHHD program will include an option for correcting an action performed, by physiologically measuring the user's dissatisfaction with an action the device was instructed to perform.

For example: When carrying out instructions received by pressing BioHHD keys, on perceiving a physiological pattern of user dissatisfaction, e.g. a rapid change in GSR waves, the BioHHD will immediately ask whether the user would like to repeat the action, using a dialog box or a voice message.

5. Using the BioHHD as a Personal Biofeedback Device

The BioHHD, whether alone or using other home appliances connected to it, can serve as a personal biofeedback device.

According to biofeedback principles, the device will provide feedback for physiological signals received from the user, by using the visual and auditory features of BioHHDs and home appliances, e.g. rising or falling light intensity or sound frequency. This will provide users with feedback concerning their physical responses to their desires, and allow them to progress toward their desires in an open monitoring loop.

Example: A complex behavior involving several bodily systems, some voluntary and some autonomous, such as eating (e.g. a diet), can in this way be provided with a visual expression, which will act as an open monitoring loop leading in the desired direction. In such a case, a user who wishes to achieve a desired physical state, such as relaxation, which will prevent undesired eating, can use the BioHHD along with another appliance connected to it—such as a virtual music drive in a connected laptop computer—as a personal biofeedback device in order to achieve the desired relaxation. Relaxation will help the user to avoid eating and thus reduce stress, as well as improving the efficiency of digestion. Another option is to use light squares and color combinations on the screen to learn about relaxation levels; relaxing music stored in the computer memory can also be used for this purpose.

Another example is stress. When users find that their bodies are experiencing a general stress response, they can use the device in the same way.

Using the BioHHD as a personal biofeedback device, which also makes use of other appliances around it, can be especially efficient for those suffering from complex medical problems with a behavioral component, e.g. migraine. In such a case, a user whose physiological signals attest to accumulating stress, e.g. rapid drop of the EMG and temperature indices, can take preventive action by using the BioHHD as a biofeedback device for practice, along with adjusting the visual and auditory feedback in the room, changing air conditioning temperature, closing shutters to reduce light, changing the computer screen to a calmer color, and so on.

In the same way, all users, especially those suffering from psychosomatic problems, will be provided with a personal relaxation program based on time constants. Neglecting relaxation practice for one day will cause the device to suggest such action and perform it if the user so chooses.

For certain age-defined populations, such as children and youths, it will be possible to program the biofeedback BioHHD to use interactive games—for example, video games or video clips which change according to the direction of input, or any other way of incorporating visual and auditory output which allows an open feedback loop.

6. Joint Biofeedback

One of the theoretical principles of biofeedback is the need, in addition to the measuring instrument (usually a computer), for a human participant, normally an expert such as a psychologist or a doctor, to manage the open feedback loop. In accordance with this basic principle of traditional biofeedback treatment, we suggest that this person can be anyone, not necessarily a professional, whether present at the user's location or not.

A) A Professional:

In this case, the professional can be present at the user's location or can be somewhere else, and provide the user with feedback and instructions about the desired state. For instance, a doctor whose patient has a BioHHD with physiological sensor may believe that the complaint has a behavioral cause and wish to guide the patient to a healthier lifestyle. During a medical appointment the doctor may use the patient's cellular phone as a biofeedback device, to demonstrate the patient's stress level and guide the patient as to how to reduce it in everyday life.

Likewise, a family doctor who knows that a specific patient's complaints tend to be behavioral and stem from failure to reduce stress and tension can ask the patient (whether at home or at the clinic) to check his or her physiological state when calling for an appointment. At that time the doctor can observe the patient's state by BioHHD or computer and provide feedback and instructions accordingly.

B) A Friend:

In the same way as when a doctor is involved, users can create a feedback loop with any other person who is present either physically or virtually by any channel of communication such as BioHHD or the internet. The friend can either only observe the user's data, or also be connected to a physiological channel by a cellular phone, and carry on nonverbal communication by familiar biofeedback protocols. (Nonverbal communication is used especially in family therapy, where it takes place in parallel to verbal communication).

In such cases, communication will take place by each user observing his or her own data on one half of the screen and the other's data—or that of several others, i.e. a group—on the other half. The feedback provided by each user can be incorporated by communication using another protocol, such as audio, video, text, visual symbols, etc.

A personal BioHHD can be used as a joint biofeedback device for several purposes:

1. Mutual feedback between two people, to learn about desired and actual states.

2. A means of expressing emotions, e.g. romantic ones. For example, two people who have met only through an indirect medium such as internet chat can choose to add an emotional channel to their conversation. This can serve as a method of finding partners at random by joining a subscription service which would allow a sort of initial mutual feedback informing two people of their emotional states when meeting through a communications channel. Such a meeting can also be based on physical location: subscribers to this service who meet, by chance or deliberately, can use the device to signal each other about their emotional states, as a sort of indicator of the success of the blind date. For example, positive feelings will provide both users with feedback as a red light in a transparent antenna or on the device display, negative feelings as a white light.

7. Polygraph Testing:

The BioHHD's biofeedback channels can serve as a personal polygraph, which will use physiological signals to present users with feedback on their honesty or sincerity levels in given situations. By observing a visual signal, e.g. a sequence of lights, or an auditory one, e.g. a rising and falling tone, users can learn about their level of sincerity.

If two or more people who are in contact, at the same place or remotely, choose to press the biofeedback channel during conversation, it will function as a polygraph. Just as in all previous cases, users will be able to freeze, record or print visual feedback and save it for various uses.

8. Feedback from User Groups

A personal BioHHD, especially a mobile one, can serve as a feedback device. It can be used for feedback only, or as an interactive device for open feedback from user groups.

A) Response

The BioHHD can serve as a unidirectional response device from users to the network, e.g. for polls. For example, all the subscribers of a certain cellular phone company who own BioHHDs with physiological panels can respond, in return for appropriate compensation, to a poll in which pictures of products are displayed on the BioHHD screen, for instance using internet pictures integrated with the BioHHD. This response will be faster, cheaper, and more reliable than an ordinary BioHHD poll.

B) Feedback

Feedback for various events which users attend virtually or physically, deliberately or by chance. For example, users in a shopping mall who wish to respond to a sales campaign will be compensated by identifying their account. They can key in a code and express their satisfaction level by having the device measure changes in their physiological signals.

9) Improved Functioning

In this case, the computer acts as the users' personal terminal to provide responses and feedback to the organizers of a group or event, in order to improve their performance according to the users' desires. For instance, a lecturer can obtain average indices from the lecture's entire audience and track their progress, to learn when people are ready for the lecture, when it should begin, and when it would be desirable to end the lecture or announce a recess due to flagging attention.

10. Alarm

This option will allow, according to information input by the user, emergency calling of another person or activating an alarm on another device, in case of an extreme change in the timing and intensity patterns of physiological signals. This option will be available even when the device is turned off.

For example, if a youth is assaulted or is in distress, changes in waves measured in muscles, such as GSR, will be extreme and will exceed the threshold determined for that user. Devices preset to respond to such a case, such as a parent's BioHHD, a network, or a home appliance such as a stereo system playing noisy music, will be activated and sound an alarm. The BioHHD company will be able to locate the youth geographically and provide immediate help. In the same way, people with medical problems, older people, and others can be supplied with similar alarm systems.

E. Exampel for Principales of Design: The Bio-Phone Product

A health-protecting, environment- and user-friendly BioHHD, which also functions as a personal biofeedback device for solving problems, curing illnesses, and improving health:

1. Some parts of the Bio-Phone are made of recycled material, e.g. the keyboard panel, which is of light wood.

2. The Bio-Phone is designed and constructed differently from a normal BioHHD, in order to emit lower levels of radiation than other phones.

3. The Bio-Phone is a personal biofeedback tool. With practice, its use can solve problems, cure illnesses, and improve the user's health and lifestyle.

Bio-Phone subscribers enjoy a service package:

4. Phone-based guidance and reminders to practice using the Bio-Phone, or other complementary behavior changes.

5. A service hotline providing technical support, professional and medical guidance, and help in emergencies.

6. Additional possibilities for improving health using online services.

7. Discounts for associated products and services related to health, lifestyle, or the environment.

8. Complementary group events and meetings to improve subscribers' health, e.g. nature treks

F. Operating Method of Phone or Computer

On the BioHHD or on the computer (including palm pilot), or on external device connected to them as keyboard or a mouse or a toy, there will be electrophysiological sensors (including tactile sensors). The change in the user condition will cause a change in specific software in the BioHHD or the computer. The change will be represented on the screen in audiovisual way and let the user operate it by the biofeedback principles. This software can for example represent the operating system of the computer in a specific audiovisual display as a 3D-video movie or presentation. The change in the user condition as measured by the sensor will operate the software, in a symbolic way, for example by changing an icon, or by moving any virtual sign on the screen, or in other audiovisual feedback display applications mentioned hereinabove.

G. Behavioral Patterns Monitor

The BioHHD or the computer will function as a medical and health monitor that collects, analyzes, stores, and sends data about the behavioral, physical and medical condition of the user. The data will be received from Electrophysiological sensors, outside and inside the body. The BioHHD or the computer can store this data, process it, save it, show it to the user, or to others, such as the physician. It can transfer this data in real time by phone, or when the user comes to the physician or to any other expert office, and then transfer the data or export it to another computer by cable, phone call or diskette. The BioHHD can also operate various devices due to the data collecting, such as devices in the user's body.

This monitor can supply additional behavioral data about complex health problems with medical and behavioral reasons, such as hypertension, that may be affected by the user's behavior. Because of this reason, blood pressure is continuously measured in the natural environment in special, costly equipment such as the HOLTER device. The monitor of the present invention will work in the same way, but will record other data, including psychophysical data, about the function of the body during stressful events. Thus, it will supply another source of information to the caregiver about the behavior patterns due to triggers in the subject's daily life, e.g. type of stress, reaction to daily life events, and how the subject copes with this stress, i.e. his natural relaxation habits, which the expert may use to make his diagnosis and offer a course of treatment.

H. Operating Software of a Game or a Virtual Book:

Thus may be done through an external device or integrative device. E.g. a keyboard designed as a child's toy and including sensors, or another device the user can use to operate hardware or software via phone or the computer. E.g.: read a virtual book in an integrative way, react to the story, operate scenarios of the story, and get physical feedback during the reading of the story from elements integrated in the external devices. This “virtual integrative sensing book” can be stored in another site, and from cellular Internet to the personal phone or computer downloads them by phone or.

I. Behavioral Data Card

The behavioral data recorded in the computer or in the phone, as mentioned in point #2, above can be stored on a small memory card or diskette. It can save data about the patient including his behavioral reaction to events as stress, relaxation, polygraph testing, etc. The card can be used as a personal data card that supplies data about the subject, for identification, or for medical use.

J. Development of a Unique Chip for Receiving and Screening Biological Signals and Turning these into the Form of Input.

A unique electrical circuit for the processing of signals, one that could be assimilated into any home consumer apparatus. This circuit screens, processes and amplifies the physiological signals coming from the chip, the chip having the possibility of being adapted to each of the apparatuses that will be mentioned below. This device could, for instance, increase the signal achieved from the measurement, and perform screening of noise before any handling of the signals is taking place. The consecutive handling of the measured signal consists of turning the datum into a digital one, converting it into serial information and integrating it into the information being transferred from the chip to the application. For instance, the technology transfers all of the measured information to the application through the standard communication channel by which the remote-control unit transfers the communication data to the air-conditioner.

K. Adding Biofeedback Sensors to an Existing Remote-Control Unit.

Exchanging the sensor of temperature in the remote-control unit of the air-conditioner with a sensor which is suitable also for biofeedback systems, and assimilating other biofeedback sensors, such as the pulse of the heart, GSR, BVP, into it.

L. Adjusting the Air-Conditioner to User Perceptions by Integrating Sensory Input of the User into the Remote-Control Unit of the Air-Conditioner.

Adding any physical input of the user will allow refining the measurement of the user's actual satisfaction from the air-conditioner operation. This is done by adding input regarding the humidity and temperature of the skin, to the calculation of suitability of the room temperature as it is expressed by the physiology of the body, and mainly by the psycho-galvanic reflex. This calculation will take into account an averaging of GSR measurement, over passing time and also in comparison with the time since turning the air-conditioner on—from the points of view of level and of rate are no more of the rapid measure of change as before, and is significantly different than the first measurement, together with a similar change in skin temperature. A positive response to both of these examinations will allow regulating the air-conditioning, while a negative response will necessitate changing the temperature and the humidity level created by the air-conditioner, respectively. A game may be played at the time of the examination being performed, or the remote-control unit can be used as a means to supply information such as user data, advertisements, etc.

M. Physiological Input from the User for the Purpose of Operating the Air-Conditioner through Additional Remote-Control Units:

The air-conditioner could also accept input, according to which it should adjust itself to the perceptions of the user, from additional input accessories, for instance, sensors assimilated into furniture, clothes, a cellular phone, Palm Pilot or a Personal Computer, and in different ways—for instance, wired or wireless communication, short-range, “blue tooth”, electricity lines, the Internet, and more. Thus, for instance, the sensors to operate the air-conditioner can be assimilated into various non-routine accessories, and even in such a way that these start operating with no need for the user to pay any special attention, while performing routine activities as has been detailed in the introduction lines to this Article, for instance, in the armrest of a chair, in clothes, in the carpet, in the home door handle or on the refrigerator door, in the car steering wheel, and so forth.

N. Using the Air-Conditioner Remote-Control Unit's Display for Showing Physiological Information

The received data, can be displayed directly on the air-conditioner's remote-control unit, as routinely essential physiological data, for instance, the body temperature, pulse, etc. It will be possible to display these data either alpha-numerically or graphically, to compare them with previous data together with a general indication of the condition, or to automatically alert a remote station or a human response. These data could be accumulated and transmitted as the need may be.

This application could run in the background, as a software that would constantly gather data about the user, analyze them and update its statistical data base.

O. Biofeedback by Consumer Product

The user could utilize the visual or audio-visual displays on the remote-control unit or on the air-conditioning unit for the sake of performing feedback. For instance, the little lights on the air-conditioner's compressor can be used as a means for giving feedback. Another possibility is to use the air-conditioner's remote-control unit. For example: the air-conditioner remote-control unit could be made of a transparent plastic material, with small lights within it. The user's condition is indicated by the color of the specific light, which appears. The same procedure could take place even when the air-conditioner is not operating.

P. A Universal Remote-Control Unit

It will be possible to make use of the developed component (remote-control unit with sensors) as a remote-control device—not only of the air-conditioner, but also of all the home apparatuses. In addition to regular control of the operation of all the home apparatuses, through keyboarding, this device will make it possible to also control them through physiological input. This input could show whether the user is in a positive or negative stress condition, to indicate this condition on the remote-control device, and offer how to change the operation of the home apparatuses from among a collection of scenarios of the operation of these home apparatuses, that it will have learnt from the uses that had been adapted in advance for such a condition. Thus, in a positive stress condition an exciting scenario, such as sports activity or TV romantics, lighting etc. might be offered.

Q. Adapting the Panel of the Air-Conditioner Compressor to take the Form of a Landscape Picture that will Adjusts Itself to the Condition:

By adjusting tiny fluorescent lights, located on the compressor panel of the air-conditioner, to the feeling of the user, it becomes a picture that changes in a general or detailed manner according to the input. In such an output, video and audio abilities can also be integrated. Thus, for instance, the air-conditioner panel can be converted to a display-screen, which includes permanent components of landscape pictures, which change every time according to the user's perceptions, accompanied by suitable music.

R. Interactive Interface for the Refrigerator

All that was said in the former paragraphs of this chapter (1-8) would also be true for the home refrigerator. With a refrigerator, it will also be possible to use means assimilated into it for the purpose of giving the feedback, such as display-screens and means of communication, in order to communicate with a remote station for transferring, processing, consulting or alerting—for instance, taking advantage of the Internet.

For example: before opening a refrigerator it will be possible for the person opening it, through a sensor assimilated into the door handle, to reduce the pressure, and this act will be indicated by feedback from lights or other sorts of display located on it. Thus, only when the person is feeling relaxed will the color of the tiny light bulbs or of the panel change to green, and then the person will open the refrigerator's door when he is more calm, and will consume less food.

S. Interface for Regulating Closed Areas—a Biorhythmically Wristwatch

Assuming that the psycho-galvanic reflex, as it is measures by measures such as GSR, ASR, etc., is the optimal criterion of a person's satisfaction—not only satisfaction from his inner environment but also from the outer surroundings, it is possible to expand its application to additional environments, as described in the above paragraphs. This expansion would be mainly to environments where there are many and complex outer pressures in existence, such as in an airplane. In order to adjust the surroundings to the ideally desirable condition for the passenger, it is possible to use the measurement of the psycho-galvanic reflex of the individual as a measure of his satisfaction from the physical climate, as well as the service quality aspects of the services given aboard the airplane. For this purpose, the means of input, processing, transmitting, and feedback must be adjusted to the limitations of these environments from the points of view of size, safety, and ease of use. These sensors could be located on a computerized personal accessory, a regular sensor being connected to the local communicational system or a sort of a bio-rhythmical watch.

The bio-rhythmical watch is a multiple-use accessory that will be supplied to passengers upon their boarding the airplane or, for instance, to hospitalized patients upon their reception into the hospital. This is a watch that includes sensors and line-or wireless broadcasting devices. The sensors would be those used in electro-physiology or biofeedback, such as the ones for checking the pulse, sweating, temperature, and even physiological measures such as blood-pressure, body heat, blood oxidization and the like—and with the. Within this watch there will be an “Environmental Conditions Processor”, which would process the data, and a line or wireless transmitter with an infrared protocol, which would transfer the data. The data transfer can be targeted to a service-giving person like a stewardess aboard an airplane, a nurse in the hospital, or in a hotel's Room Service, to whom it will transmit a signal of prominent dissatisfaction. In another form of data transfer, the data could be targeted at a control system of an apparatus, such as an air-conditioner. The air-conditioner will adjust the outgoing-air vent personally, until a satisfaction measure has been achieved for the specific user. Another application of the watch is in choosing the desirable piece of music, the movie title, or an Internet site, in an individually adapted viewing device.

Following are two applications of this device:

1). A Bio-Rhythmical Detector for Children or Helpless Persons

The design of this detector is done in the form of a bracelet or a pin, which are attached to the clothing/the diaper and include the mentioned sensors. It can also be styled in another form, in which it would integrate in existing accessories, such as a blanket, or within an accessory such as a gaming-glove, a doll, and more . . .

The detector constantly transfers signals about the user's condition to the control system or the air-conditioner, respectively. This bio-rhythmical detector will identify an excess of coldness or warmth during sleeping, and will enable taking action to eliminate the problem by changing the room temperature, or by covering or removing the blanket.

The same signals could also be used for sending an alarm of vital signs. Alternatively, it can be connected to existing medical sensors, and process their data for the sake of adjusting the air-conditioning level.

2). Control and Feedback Device for an Airplane

As mentioned in paragraph 6 above, this input device can be used as a control means on air-conditioning or the service in an airplane, and to operate machines and computers as detailed in the former paragraphs. For example: operating of games on the passenger's personal watching-screen, according to the principles which have been detailed in the former paragraphs, or with input means detailed in those paragraphs. Thus, for instance, it will be possible to reduce stress; anxiety or even treat flight-phobia, to perform scans, to call for service when in a condition of dissatisfaction, etc.

T. Vehicles

The same principles can also be applied to cars, i.e. the assimilation of the sensors everywhere the user's hands touch—such as the steering-wheel cover, armrest, BioHHD mount, or portable or “palm” computer, or a unique input accessory. The applications would be as for all former 25 sections, and, for instance, adjusting the air-conditioner of the car to the user's feeling, a local bio-feedback apparatus that would not allow traveling until pressure decreases, choosing radio-stations or music or interior lighting according to this feeling, etc.

U. Closed Environments

As said in paragraph 6, this input device could be used as a control-means for air-conditioning or for service in any closed environment, such as inside vehicles, public institutions like a hotel, aboard a vessel or in an hospital, as well as any other place where many people are staying together simultaneously for relatively longer periods of time, i.e. for over an hour.

V. Reduction of Stress, Biofeedback, or Operation of Appliance using the Earphone of a Cellular BioHHD:

1. An earphone of a cellular BioHHD is any device that includes an earphone that is connected with or without a wire to a cellular BioHHD, whether it is independent or integrated into another device, such as a portable computer, palm computer, watch, and the like.

2. The earphone can be a device that is attached to the ear by holding it in the hand (such as a cellular BioHHD mouthpiece), or an earphone that is fitted over the ear in a manner that the hand is not necessary for support. It can be connected to a plastic strip that is placed on the head with or without a microphone attached to it.

For example: A stiff and wide plastic strip placed on the head, and supported in the area of the temporal artery on the head. An earphone and microphone are connected to this strip.

1. A sensor is attached to the earphone. The sensor can be ergonomically inserted in the earphone or, outside it and connected to it with a wire or wirelessly, for example in the shape of a clothespin worn over the ear with the sensor inside.

For example: A microphone and earphone attached to a plastic strip, including an ox metric sensor and a pulsating sensor. The strip is built such that its center of gravity is on the temporal artery between the ear and the eye. The sensors are inserted in the center of this pulse.

2. The sensors that are integrated in to the earphone can be any one of the acceptable sensors in electro physiology in a polygraph or biofeedback, such as bvp, respiration, heart rate, ecg, gsr, skin temperature, emg, oximeter.

3. The processing of the input from the sensor will be done with a chip implanted within it, or by BioHHD, or computer, or remote station as detailed in the previous section of this application.

4. The earphone allows the action of measuring biological signals such as heat, pulse, and improvement in behavioral-medical status, reduction of stress or biofeedback.

5. The abovementioned is performed via conscious or unconscious change in indicators such as skin moisture, skin electrical charge, blood oxidation level, blood flow velocity, pulse wave, and heartbeat.

6. This change will be performed via audiovisual feedback via the BioHHD itself, or another device.

For example: A status of stress that is relatively higher than personal average will be expressed at the beginning of the exercise by a slow and especially weak musical rate, or in the form of an appropriate led display on the BioHHD monitor or via another device, and afterwards changes according to the exercise as detailed in the previous two applications submitted by me, as below mentioned.

Additional example: The pulse wave velocity that is measured in the oximeter attached to the temporal artery is the input for the auditory biofeedback reduced by the user's appropriate mental action.

1. The data of the sensorial input can be transmitted or stored in the BioHHD itself or the remote station, and serve as a base for accumulative measurement for the purpose of input comparison.

2. The data can be transmitted to a specialist doctor that will operate according to the comparison of received pulse wave data, and relative to the accumulative personal average of the patient, he can render consultation or call for assistance, according to need.

Additional example: Measurement of temperature in the ear, and comparison to personal average.

1. These data may also be applied to other purposes, as detailed in the previous two applications and the previous sections of this application. For example, the application that is detailed in this application is an air-conditioner. The sensorial input implanted in the earphone can be processed within it, or a BioHHD, and be transmitted to the user's office or home. From there, it is transmitted over the home communicational system or Internet, or electrical lines to home appliances. The status of the user, relative to his personal average, constitutes an input for a “universal remote control” for the activation of home appliances, according to this sensation. In the example of the air-conditioner, a stress level that is higher than average can prepare, prior to his entry into the house, via BioHHD transmission, the temperature level, moisture, and wind direction of the air-conditioner in the scenario that is appropriate to a high stress level, as preprogrammed.

Additional example: The driver of a vehicle has the option of adjusting his air-conditioner or radio according to sensorial input, for example blood oxidation level.

Y. Additional Applications

Additional applications of the principles detailed in paragraphs 1-13, such as applications for the assistance of the earphone in par. 13 for handicapped, controlled adjustments of homogenous work-environments, medical application assistance of detectors unique for that purpose

X. Cradio Analysis Application

Studies have shown that a few simple breathing exercises practiced on a regular basis may improve the condition of heart attack victims. The present invention provides a method to treat patients using these breathing exercises and a biofeedback device.

The method and device may also be used by others, and after being used for a period of time the device will indicate whether the user shows symptoms or warning signs of heart problems. The process of using this method is described below.

The biofeedback device is easy to use, and may be connected to an electronic hand held device with a display such as cellular phone. The user must hold the device with his/her fingers touching the sensors. Then the user plays an interactive game, which incorporates the various breathing exercises. Throughout the game, the sensors measure data such as the user's pulse, heart rate, or blood pressure. This data may be displayed on the screen as the user plays the game. The game itself may also alter in accordance with data received by the sensors in order to optimize comfort, fun, and progress.

Throughout the interactive game the device records all data received as statistical information inside a database. This information is processed as a function of the BVP (Blood Volume Pulse) heart rate, BVP amplitude, heart rate variability (and entrainment ratio) to estimate the user's overall heart condition. Progress is tracked over a period of time or uses, and may be viewed by the user at any time. The user is also notified if the statistics indicate a health risk that should be examined or treated. The data processing of recording received data and statistical analysis of the recorded data can be performed by the hand-held device itself or alternatively by a designated remote computer device that receives the measurements data through communication network.

HRV analysis can detect early subclinical alterations of the autonomic nervous system in asymptomatic patients with IDDM, which seem to consist mainly in a parasympathetic impairment. Autonomic dysfunction is associated both with the duration and an inadequate metabolic control of the disease.

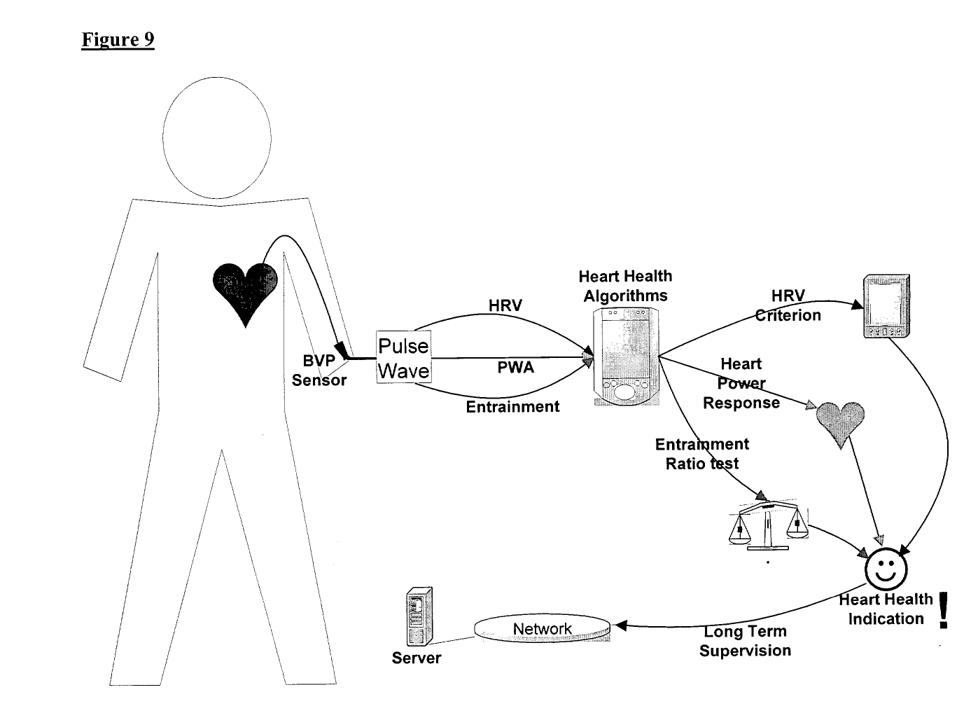

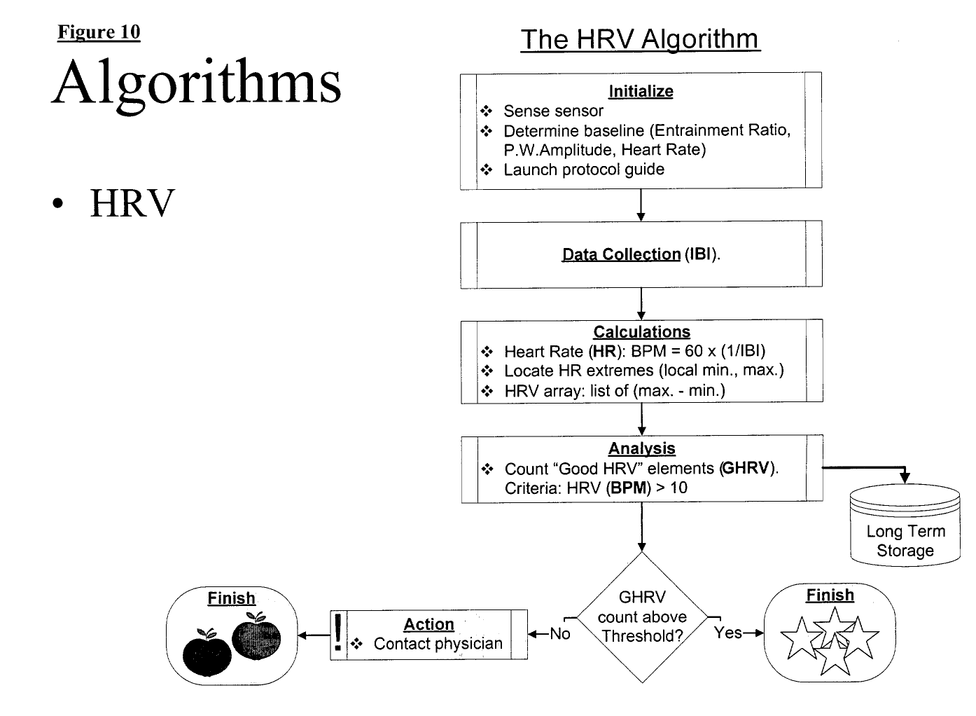

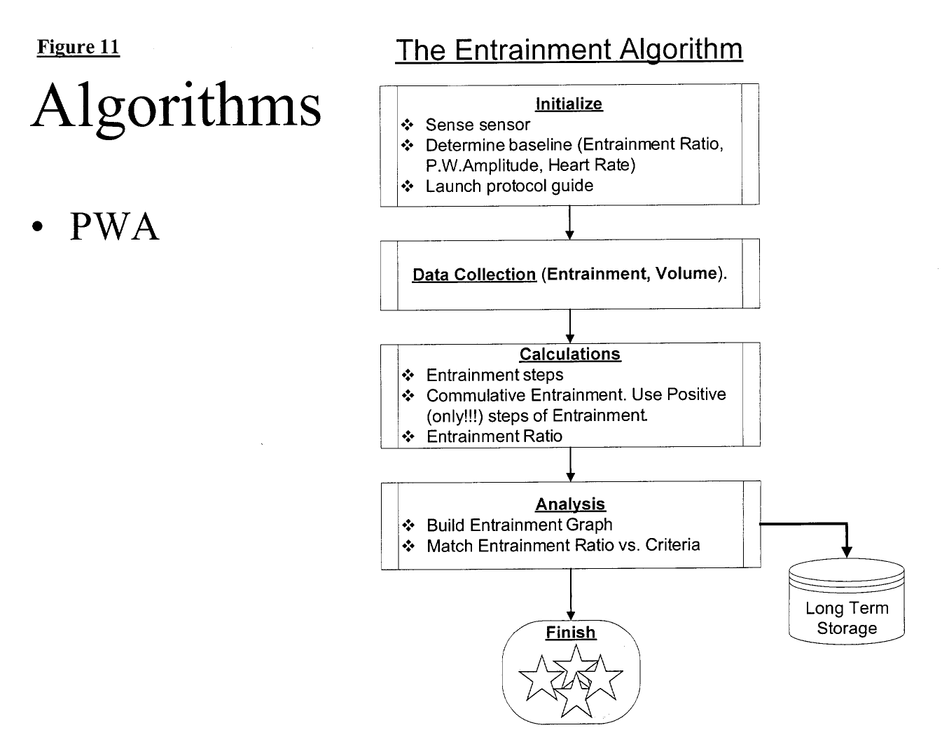

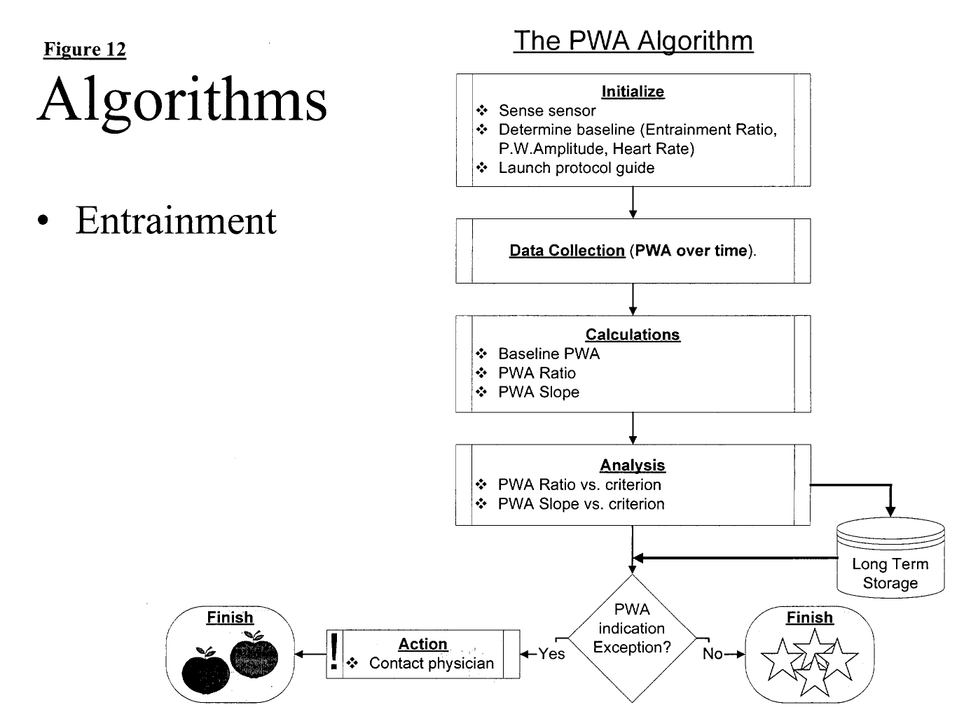

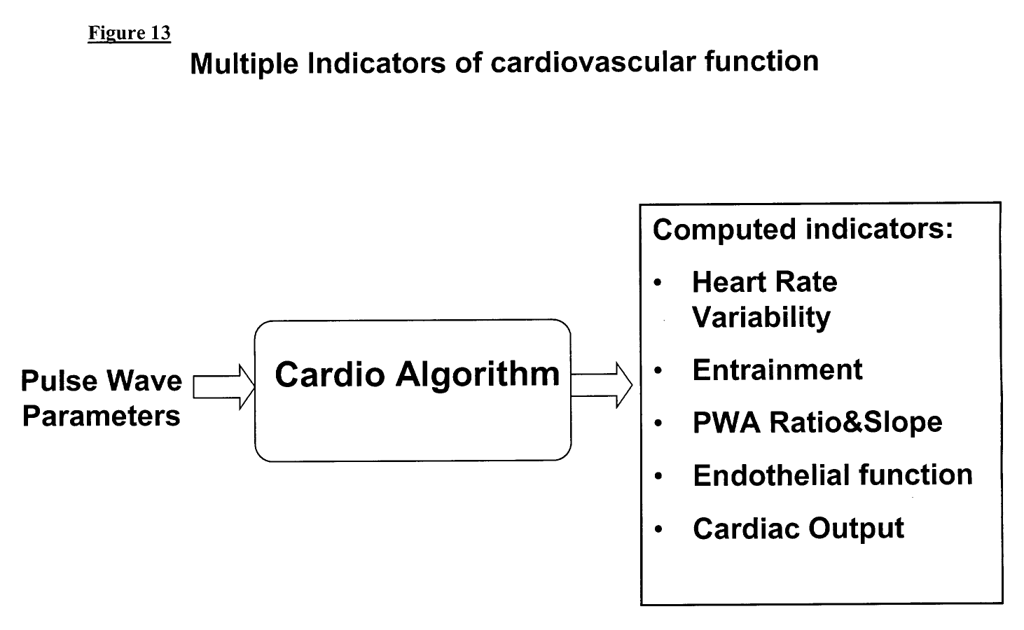

The crdio anlysis process is based on measuremnts of pulse wave which is measured by pulse oximeter sensor. The output of the analysis include comabination of several indicators as seen in FIG. 13. The cardio algotithm priciples are detailed in the FIG. 10-12 and data flow is illustrated in FIG. 9.

References

U.S. Pat. No. 6,319,205

U.S. Pat. No. 6.322,515

PCT no. WO0234105

U.S. Pat. No. 6,461,305

International patent no. WO02080752

U.S. Pat. No. 6,488,633

U.S. application Ser. No. 20030004423

Bibliography

1. Amir, M., Yaniv, N., & Paran, E.)1994). The Assessment of the long Term Effect of Biofeedback Assisted Relaxation)BAR) with a Mental Stress Test. In: Sakuma, H. (Ed.(Bio behavioral self regulation in the east and the west. pp. 577- 582. Springer, Tokyo.

2. Amir, M., Yaniv, N., & Paran, E)1996(Evaluating The Response Of Mild Hypertensives To Biofeedback Relaxation Using A Mental Stress Test. Journal Of Behaviour Therapy And Experimental Psychiatry 27(2): 157-167.

3. Katz, A., Liberty, I F, Porath A, Ovsyshcher I, Prystowsky ENA (1999) Simple Bedside Test of 1-minute Heart Rate Variability During Deep Breathing as a Prognostic Index after Myocardial Infarction. American Heart journal 138:32-8

4. Nolan R. P., Kamath, M., Young, Q. R., Pang, C., Floras, J. S. (2002) Heart Rate Variability Biofeedback: A Novel Procedure to Enhance Vegal Recovery from Acute Stress. American Psychosomatic Conference,

5. Rozanski, A., Qureshi, E., Bauman, M., Reed, G., Pillar, G., Diamond, G. A. (2001). Peripheral Arterial Responses to Treadmill Exercise Among Healthy Subjects and Atherosclerotic patients. Circulation 103: 2084-2089.

6. Yaniv, N. Paran, E. & Amir, M.)1993(The Effect of Biofeedback Assisted Relaxation on mild Hypertension. 14^{Th} International conference of Stress and Anxiety Research Society, Cairo.

7. Yaniv, N. Paran, E. & Amir, M.)1993(.Mental Stress Test as a Method to Evaluate BAR in Mild Hypertension. 6^{th} Annual Meeting of Mediterranean Association of Cardiology and Cardiac Surgery. Corfu.

8. Yaniv, N. Paran, E. & Amir, M. (1993(The assessment of the long term effect of Biofeedback assisted relaxation (BAR) with a Mental Stress Test”. 3^{Th} International Conference on Biobehavioral Self-Regulation and Health. Tokyo.

9. Yaniv, N., Viskoper, J. R., Heilbrun, S., Weitzman, S. (1994). Nonpharmacological Control of Hypertension. 5^{Th} international symposium on hypertension in the community, Tel-Aviv.

10. Yaniv, N. (2002). Aparatus of Heart Rate Variability Biofeedback for a Cellular Phone. The 7^{th} International Congress of Behavioral Medicine, Helsinki.

11. Yaniv, N. (2002). Cellular Phone as a Personal Biofeedback Device. The 4^{th} world Congress on Stress, Glasgow.

12. Yaniv, N. (2002). Wierless Gaming and Health: A new Methodology. The 5 Th. European Conference on Gambling.Barcelona.

13. Yaniv, N. (2002). Stress and Anxiety reduction Before Anesthesia by a mobile phone. the 8^{th} International Congress of Cardiothoracic and Vascular Anesthesia in conjunction with the 19th Congress of the Israel Society of Anesthesiologists, Tel Aviv.

14. Yaniv, N. (2002). E-Gambling and health: a new metodology for population studies. The 13^{th} Annual Meeting of the American Academy of Addiction Psychiatry, Las-Vegas.

15. Yaniv, N. (2003). Biofeedback in a personal computer mouse. 6^{th} international conference on stroke & 3^{rd} conference of the Mediterranean stroke society, Monte-Carlo.

16. Yaniv, N. (2003). Interactive Gambling on a Mobile Phone. The 12^{th} international Conference on Gambling and Risk Taking, Vancouver.

What is claimed is:

1. Electro-physiological sensors assimilated into the structure of an electronic device, said electro-physiological sensors constantly measures physiological signals from the user skin surface, wherein the signals are analyzed and processed in real time, generating real time feedback indication and instigating an operation electronic device which affects the interaction between the user and said electronic device in accordance with the measured physiological changes.

2. The device of claim 1 further comprising a communication interface for transmitting measurement data signal via communication network to a designated computer device that analyses the measured data and returns response data or content.

3. The device of claim 1 wherein the physiological signals are measured from the user finger;

4. The device of claim 1 wherein the physiological signals are measured from the user temple;

5. The device of claim 1 wherein the electronic device includes wireless communication means.

6. The device of claim 1 wherein the electronic device functions as an interface to computer apparatus.

7. The device of claim 6 wherein said interface is computer mouse device.

8. The device of claim 1 wherein the electronic device functions as an interface to electrical appliances and the feedback operation affects/controls the operation of the electrical device;.

9. The device of claim 8 wherein said interface controls operation of air conditioner and said air conditioner temperature is controlled according to physiological measurements received from the user.

10. The device of claim 8 wherein said interface controls the operation of television apparatus and the television channel selection is controlled according to physiological measurements received from the user.

11. The device of claim 1 wherein the feedback operation's output is a visual and or audio indication of user mood

12. The device of claim 11 wherein said feedback operations are part of computerized game.

13. The device of claim 1 wherein the feedback operation is associated with network communication services enabling the selection and retrieval of relevant content in accordance with user's mood.

14. The device of claim 1 wherein the feedback operation is associated with broadcasting communication services enabling the selection and retrieval of relevant content in accordance to user mood.

15. The device of claim 1 wherein the feedback operation is associated with network communication services of interactive sessions between at least two users enabling to change or affect transmitted messages between the users and to enable one user to receive real time physiological data of a second user indicating second user's mood, feelings or health condition;

16. The device of claim 1 wherein the feedback operation is indication of polygraph analysis in accordance with measured physiological parameters

17. The device of claim 1 wherein the feedback operation is associated with polls research processes wherein the poll results are measured in accordance to measured physiological parameters;

18. The device of claim 1 wherein the feedback operation is associated with vehicle operation alerting the user when identifying sleeping or stress states;

19. The device of claim 1 wherein the feedback operation is based on analysis the medical condition of the user according to sample measurements of blood flow and heart function enabling to detect abnormal situation and alert the user;

20. The device of claim 19 wherein the sampled measurements are recorded and the analysis of medical condition includes estimation of the user heart health condition according to statistical results of collected measurements;

21. The device of claim 20 wherein the data recording and analysis is preformed at remote computer device;

22. The device of claim 19 wherein electronic device includes wireless interface and the feedback operation further enabling to send alert messages to medical emergency service;

23. The device of claim 19 wherein the physiological measurements are received as feedback to interactive game session;

24. The device of claim 19 wherein the blood flow and heart function include blood volume pulse, pulse wave amplitude, power spectrum, blood pressure, heart rate variability, entrainment, cardiological output, heart rate, bit per minute, arterial tone, pulsatile blood volume and finger arterial pressure.

25. The device of claim 19 intended to be used for non clinical purposes.

26. The device of claim 19 intended to be used for clinical purposes.

27. The device of claim 1 further comprising physiological sensors embedded within earphone device, said earphone device associated to electronic device for receiving physiological measurements;

28. The device of claim 1 wherein feedback operation uses the physiological measurements as control parameters of a games of chance influencing the risk factor of the game.

29. Sensors for measuring cardio-vascular activity from the user skin surface assimilated into the structure of an electronic device, wherein the measured signals are sampled, recorded and processed in real time , wherein said processing include statistical analysis and cardio analysis of the recorded data resulting alert report based on detecting abnormal situation.

30. The device of claim 29 further comprising a communication interface for transmitting measurement data signal via communication network to a designated computer device that analyses the measured data and returns response data or content.

31. The device of claim 29 wherein the physiological signals are measured from the user finger;

32. The device of claim 29 wherein the physiological signals are measured from the user temple;

33. The device of claim 29 wherein the electronic device includes wireless communication means.

34. The device of claim 29 wherein the electronic device functions as an interface to computer apparatus.

35. The device of claim 34 wherein said interface is computer mouse device.

36. The device of claim 29 wherein the electronic device functions as an interface to electrical appliances and the feedback operation affects/controls the operation of the electrical device;.

37. The device of claim 36 wherein said interface controls operation of air conditioner and said air conditioner temperature is controlled according to physiological measurements received from the user.

38. The device of claim 29 wherein the measurement process are part of computerized game.

39. The device of claim 29 wherein the data recording and analysis is preformed at remote computer device;

40. The device of claim 29 wherein electronic device includes wireless interface further enabling to send alert messages to medical emergency service;

41. The device of claim 19 wherein the cardio vascular measrements include blood volume pulse, pulse wave amplitude, power spectrum, blood pressure, heart rate variability, entrainment, cardiological output, heart rate, bit per minute, arterial tone, pulsatile blood volume and finger arterial pressure.

42. The device of claim 29 intended to be used for non clinical purposes.

43. The device of claim 29 intended to be used for clinical purposes.
